(19) United States
(12) Patent Application Publication
(10) Pub. No.: US 2008/0109077 A1
(43) Pub. Date: May 8, 2008

(54) Intraocular Lens

(75) Inventor: Gilles Bos, Onex (CH)

Correspondence Address: YOUNG & THOMPSON, 209 Madison Street, Suite 500, ALEXANDRIA, VA, 22314, US

(73) Assignee: XCELENS S.A., Geneve (CH)

(21) Appl. No.: 11/547,633

(22) PCT Filed: Apr. 4, 2005
(86) PCT No.: PCT/FR05/00809

§ 371 Date: Oct. 5, 2006

(30) Foreign Application Priority Data
Apr. 6, 2004 (FR) 0403615

(51) Int. Cl. A61F 2/16

(52) U.S. Cl. 623/6.43; 623/6.49; 623/6.51

(57) ABSTRACT

The invention relates to an improved intraocular lens. According to the invention, the haptic (3) support part (4), elevation view, takes the form of a flat, closed, semi-circular loop having a constant thickness at rest. The proximal end of the loop extends from the distal part of a connecting stalk (5) which extends essentially tangential to the periphery of the optical part (2) and in parallel with the vertical diameter (d1) of the optical part. The haptic (3) support part (4) forms an angle of approximately 30° with the horizontal diameter (d2) of the optical part (2).

The invention relates to intraocular lenses and more especially the problem of centering the lens in the eye, both in the capsular bag as well as outside of said bag. The object of the invention is to provide increased refractive stability and predictability to the lens with a reduction of risk of cellular migration for the lenses that are designed to be implanted in the capsular bag.

It is known that an intraocular lens consists of an optical portion, designed in use to be centered on the optical axis of the eye, and a haptic portion, connected to the periphery of the optical portion whose role is to maintain the centered position of the latter by enclosing the deformations undergone by the eye during the process of accommodation and aging thereof. Because of this essential function, the haptic portion of an intra-ocular lens should meet numerous constraints:
it should be able to deform in the optical plane,
it should be substantially nondeformable in the optical axis,
it should not transmit any force likely to deform it to the optical portion,
it should, in use, have the largest support surfaces possible,
it should be foldable for injection into the eye through a small self-suturing incision on the order of 3 mm,
it should neither injure the eye nor be a factor of post-operative complication.

For about 10 years, numerous solutions have been proposed, and a large variety of forms of haptics have been seen to emerge both at the level of their support portion as well as their connecting portion to the optical portion.

Thus, patent U.S. Pat. No. 5,716,403, which relates to a one-piece flexible lens, proposes open-loop haptics with a connecting portion that has a smaller thickness, on the front side, than that of the support portion and an elbow of this support portion above the connecting portion. This lens, however, exhibits the drawback of a relatively high rate of opacification of the capsular bag in comparison to the preceding generation. Actually, the design of its haptics concentrates the entire deformation on the minimum section point, but is overall too rigid, which does not allow it to adapt to the different shapes of patients' eyes and causes a capsular fold that promotes the cellular proliferation in patients having small eyes. In contrast, the weakening of the axial thickness of the haptic at the connecting portion brings about a risk of imbalance in the two ansae and an alternation of the optic that the surgeons refer to by the name “Z Syndrome.” Finally, the large width of the connection portion, which extends over an angular sector of about 45° at the periphery of the optic, constitutes, because of the absence of inclination of the haptics relative to the optical plane, a springboard for the epithelial cells that interfere between this connection portion and the rear capsule of the capsular bag and thereby can proliferate in the direction of the optical axis.

The patent WO 97/41 805, also relating to a one-piece flexible lens, proposes closed-loop haptics with a connection portion that is even larger than that of the lens of patent U.S. Pat. No. 5,716,403, encompassed between 60° and 90° of the angular sector, and reduction of the thickness of the haptics in the connection zone. This lens therefore exhibits the same design defects as the preceding lens.

The flexible lens that is described in WO 00/53124 comprises haptics that consist of support portions that are triangular as a whole and that are connected to the optical portion by two arms. The fixation points of said arms to the optical portion are spaced from one another; consequently, the connection zone of each haptic in the optical portion extends over about 90°. This lens therefore exhibits the drawbacks that are described above.

A flexible lens whose haptic portions are in the shape of a split loop so as to constitute hooking clamps of the lens to the stationary portion of the eye is also known from US 2003/0195622. The implementation of such clamps increases the risks of injuring the eye receiving said lens.

This invention proposes a new form of haptic for an intraocular lens that is characterized by the fact that its support portion, elevation view, has the shape of a flat, semi-circular closed loop with a constant thickness at rest, the proximal portion of said loop originating on the distal portion of a connecting peduncle that is oriented essentially tangentially to the periphery of the optical portion and parallel to the vertical diameter of said optical portion, whereby the support portion of said haptic forms an angle of about 30° with the horizontal diameter of said optical portion. This particular shape of the support portion of the haptic, which can be compared to that of an elastic bracelet, exhibits the capacity of deforming into an infinity of overall circular shapes, which, in its application to an intra-ocular lens haptic, ensures uncompromising adaptation to the morphology of the patient's eye and a drastic reduction of the projection forces along the optical axis created in the lens during its installation, consequently the compression of the haptics along the optical plane of the lens.

According to an advantageous characteristic, the bracelet that forms the support portion of the haptic has a general arc shape with an aperture angle of said arc of between 30° and 60°, and preferably 40°.

The bracelet also has the following advantageous characteristics:
it has a section that is constant over its entire length,
its current section is between 0.05 and 0.50 mm², preferably 0.20 mm²,
the width and the thickness of the thread or strip that forms the loop are between 0.2 and 0.8 mm, preferably 0.4 mm,
the width of the bracelet is between 0.6 and 3 mm, preferably 2 mm, and its length is at least equal to twice its width.

Other characteristics and advantages of the invention will emerge from the following description of two embodiments of an intraocular lens of a capsular bag, equipped with haptics according to the invention. This description, given by way of non-limiting example, will refer to the accompanying drawings in which:

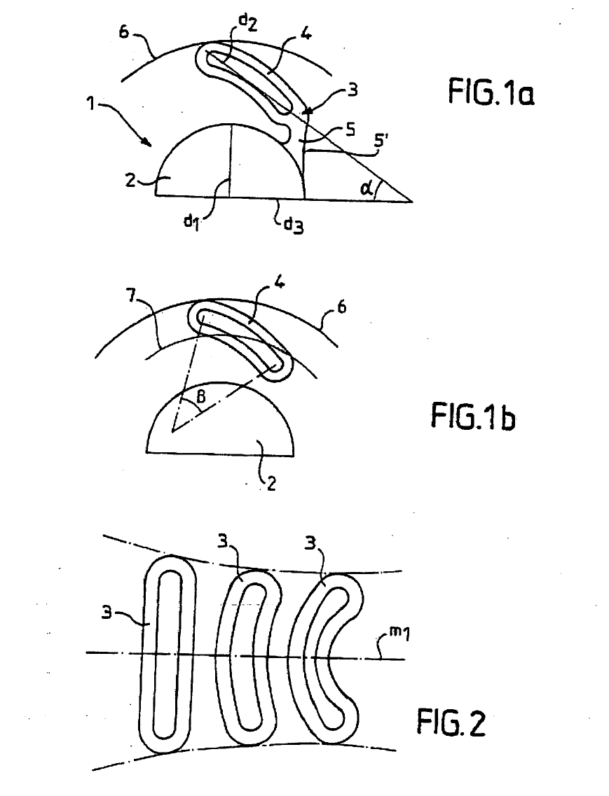

FIGS. 1a and 1b are schematic diagrams of a first embodiment of the invention over a half-plan view of an intraocular lens,

FIG. 2 is a diagram showing the principle of the circular deformation of the bracelet loop of the support portion of the haptic according to the invention,

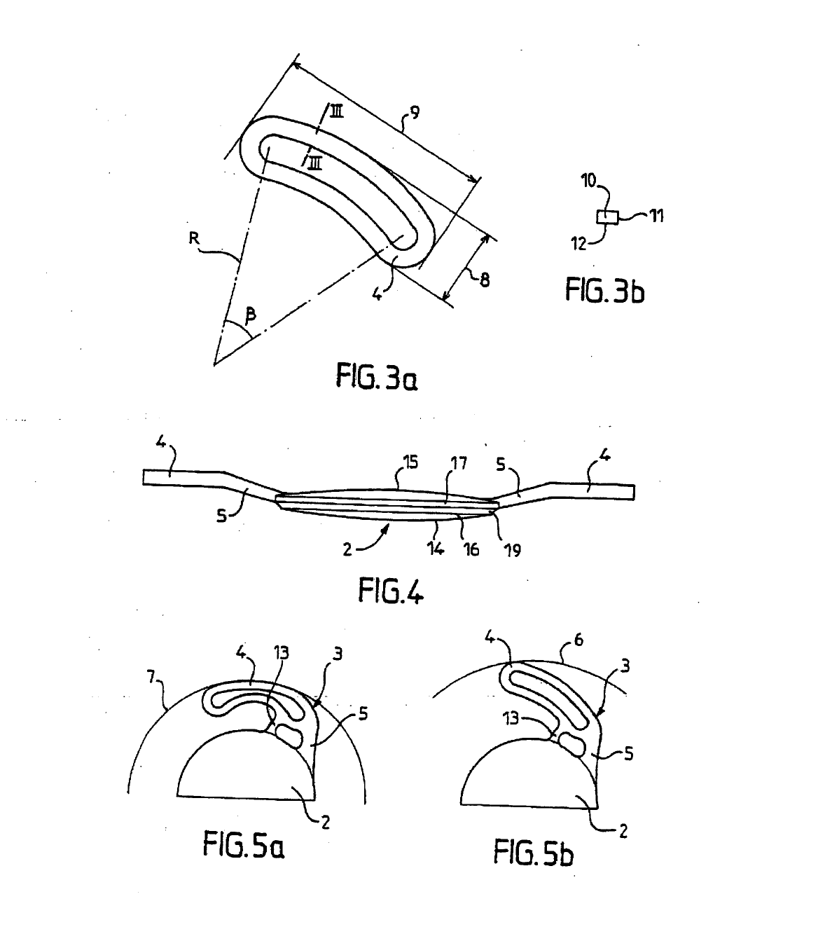

FIG. 3a is a diagram that illustrates the general characteristics of the support portion of the haptic according to the invention,

FIG. 3b is a cutaway view along line III-III of FIG. 3a,

FIG. 4 shows diagrammatically the connection to the optical portion of an intraocular lens of the support portion of the haptic according to the invention,

FIGS. 5a and 5b are schematic diagrams of a second embodiment of the invention on a half-plan view of an intraocular lens, whereby 5a is the view of the lens in its compressed form (inside the eye), and whereby 5bis a view of the lens before its introduction into the eye,

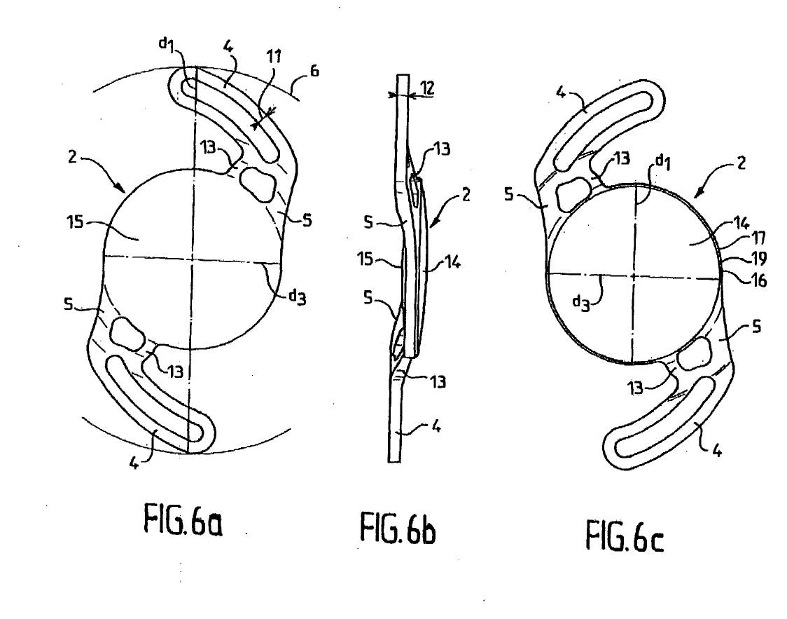

FIG. 6a is a view of the front face of a one-piece lens with a capsular bag produced according to the diagrams of FIGS. 5a and 5b,

FIG. 6b is a profile view of the lens shown in FIG. 6a,

FIG. 6c is a view of the rear face of the lens shown in FIGS. 6a and 6b,

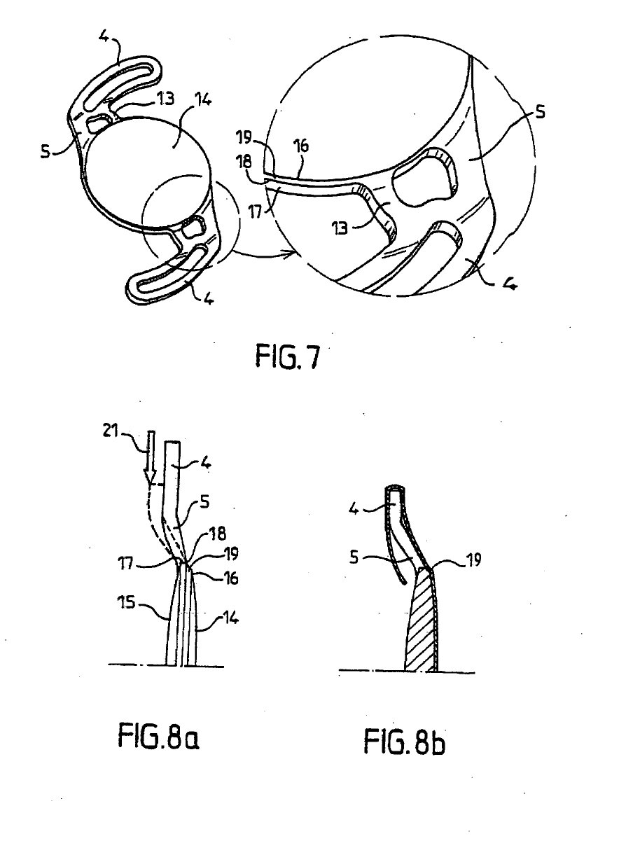

FIG. 7 is a perspective view with partial enlargement of the lens shown in FIGS. 6a, 6b and 6c,

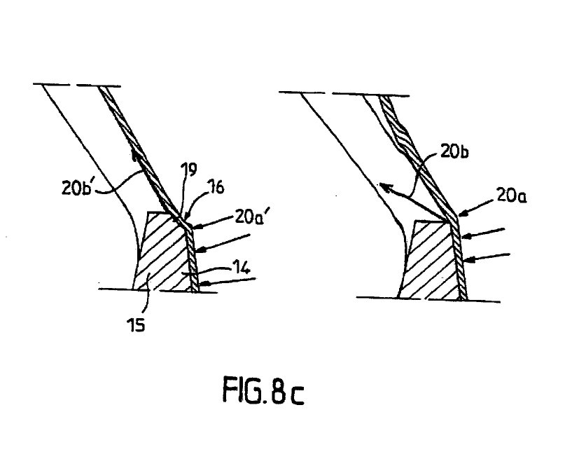

FIGS. 8a and 8b diagrammatically illustrate the projection of an intraocular lens haptic according to the invention before (FIG. 8a) and after (FIG. 8b) its installation in the capsular bag.

FIG. 8c is a comparative diagram showing the behavior in the capsular bag of a lens of the prior art and of the lens according to the invention at the connection of the haptic portion to the optical portion.

FIG. 1a shows a diagrammatic half-view of the front face of an intraocular lens 1 with its overall circular optical portion 2 and its haptic portion 3 that breaks down into a support portion 4 and a connecting portion 5. The circular line 6 shows the inscribed circle that is defined by the lens 1 at rest. FIG. 1b shows only the optical portion 2, the support portion 4 of the haptic 3, the inscribed circle 6 of the lens at rest and the inscribed circle 7 of the lens in use.

It is seen in these figures that the connecting portion 5 forms a peduncle that is connected to the periphery of the optical portion 2 over an angular distance that is relatively small relative to the circumference of the implant. This angular distance will preferably be on the order of 15° to 20°. It is also seen that the peduncle 5 extends in a general direction that is virtually parallel to the vertical diameter dl of the optical portion 2 and that its outside edge 5′ is essentially tangential to the periphery of the optical portion 2.

On the distal portion of the peduncle 5, the support portion 4 of the haptic 3 originates in a general direction d2 that forms an angle a of about 30° with the horizontal diameter d3 of the optical portion 2. The support portion 4 has the shape of a flat semi-circular closed loop with a constant thickness at rest, analogous to that of an elastic bracelet. In FIG. 1b, it is seen that the arc formed by the loop 4 extends over an angular distance β of about 40°.

As indicated above, this particular form of the support portion 4 of the haptic 3 has the property of deforming uniformly along infinite loops that are coplanar to the initial loop around the median axis ml of the loop, as FIG. 2 illustrates.

Thanks to these arrangements, it is ensured that the compression forces that are exerted on the haptic of the lens after its installation in the eye are concentrated on the median axis ml of the loop 4 parallel to the optical plane of the lens, which both makes it possible for the support portion 4 to assume the contour of its housing over a great length and without rough spots and to reduce the projection forces of the optical portion 2 on the optical axis.

In practice, for an intraocular lens whose overall diameter at rest (reference 6 in FIG. 1a) is on the order of 13 mm, and the overall diameter in use (reference 7 in FIG. 1b) is on the order of 10.5 mm, the following dimensions (cf. FIG. 3) will be adopted by taking as reference a plane that is perpendicular to the optical axis:

height 8 of the loop: from 0.6 to 3 mm, preferably 1.4 mm, length 9 of the loop: from 1.2 to 6 mm, preferably 5 mm,
section-10 surface in the thread or strip that constitutes the loop: from 0.05 to 0.5 mm², preferably 0.2 mm²,
width 11 of the thread or ribbon that constitutes the loop: from 0.2 to 0.8 mm, preferably 0.4 mm,
thickness 12 of the thread or ribbon that constitutes the loop: from 0.2 to 0.8 mm, preferably 0.4 mm,
initial curvature radius R of the loop: from 4.5 to 6 mm, preferably 5.75 mm.

Preferably, a strip section with a thickness 12 that is equal to or greater than its width 11 will be adopted so as to promote the homogeneous deformation of the two superposed fragments of the loop 4 and to avoid inducing torsion components that would have an adverse effect on the stability of the haptics.

The peduncle 5 will have virtually the same thickness as that of the thread or strip that constitutes the loop, either from 0.2 to 0.8 mm and preferably 0.4 mm, while its height relative to the circumference of the optical portion will be on the order of 0.4 to 2 mm and preferably 0.6 mm. These arrangements ensure that the peduncle 5 does not interfere significantly with the homogeneous deformation of the support portion 4.

In the case of an intraocular lens with a capsular bag, it is recommended to provide to the haptic portion, profile view in the optical plane, an inclination relative to said plane so as to create during the compression of the haptics a force displacing the optical portion in the direction of the window along the optical axis, which has the result of flattening the rear diopter of the optical portion against the rear wall of the crystalline bag. As is seen in FIG. 4, the haptic portion of the intraocular lens according to the invention has such an inclination at its peduncle 5, while its support portion 4 is parallel to the optical plane. According to an advantageous characteristic of the invention, the peduncle 5 of the haptic portion connects directly to the periphery of the front diopter of the optical portion along an angle of about 15° relative to the optical plane, which determines a projection toward the front of the support portion 4 at rest of about 0.6 mm relative to the edge limiting the circumference of the rear diopter of the optical portion and ensures that this projection will not exceed a value of between 0.9 and 1 mm after the installation of the lens in the capsular bag.

Thus, by the combination of the flat elastic bracelet form of the support portion of the haptic, its inclination along an angle of about 30° on its peduncle for attachment, and implantation of said peduncle on the periphery of the front diopter of the optical portion with an inclination of about 15°, the intraocular lens according to the invention produces the following performances:
a flexion of the support portion contained in a plane that is perpendicular to the optical axis, with a component of limited projection,
a very homogeneous flexion of the support portion that is in perfect conformation with the compression environment: this support portion behaves in the opposite way from the main window (between the two fragments of the bracelet) when it flexes, which provides it with the capacity not only to adapt to the dimensions of the eye that receives it but also not to produce capsular folds,
a projection induced by the limited compression of haptics: a distance of more than 0.9 mm between the edge of the rear diopter of the optic relative to the rear plane of the support portions of the haptic makes it possible to obtain a projection force that ensures the contact of the rear diopter with the rear capsule,
an acute-angle contact of the peripheral edge of the rear diopter with said rear capsule, amplified by the production of an embedding of this optic in said rear capsule, an embedding that is linked to the projection force, itself linked to the structure of the inclination and to the presence of this acute angle,
an absence of “Z Syndrome” or decentering of the optical portion, as well as a prevention of the cellular proliferation responsible for the capsular opacification.

FIGS. 5a and 5b represent an advantageous variant embodiment of a capsular bag implant according to the invention. It is seen in these figures that a strut 13 designed to improve the deformation homogeneity of the support portion 4 was added under the support portion 4 of the haptic 3 and close to its proximal end, essentially parallel to the peduncle 5. By its position, this strut 13 avoids any flexion of the support portion 4 that would not be due to a deformation of the latter as well as any flexion of the peduncle 5.

FIGS. 6 to 8 illustrate a preferred embodiment of an intraocular lens according to the invention. In these figures, the reference numbers refer to explanations provided in relation to the preceding figures. In FIGS. 6a, 6b and 6c, the same essential constituent elements as in the FIGS. 5a and 5b are found, namely: the optical portion 2, circular and biconvex, the connecting peduncle 5 implanted on the periphery of the front diopter with an inclination of about 15°, the support portion 4 in the form of a semi-circular bracelet attached to the peduncle 5 in a general direction forming an angle of about 30° with a plane passing through the horizontal diameter of the optical portion, and the strut 13 that is essentially parallel to the peduncle 5 and placed under the proximal end of the bracelet 4.

The lens 1 exhibits a particular configuration of the periphery of its optical portion, designed to reinforce the prevention against the cellular proliferation. Actually, the diameter of the rear diopter 14 is smaller than that of the body 15 and the connection between the edge 16 that is thus created and the narrow peripheral edge 17 of the optic is made by a concave neck mold 19 that defines a second edge 18 with said narrow edge. Said edge 18 also extends over the entire periphery of the narrow edge 17, including in the connecting portions of the peduncles 5 and struts 13. In addition, the implantation of peduncles 5 and struts 13, whereby they are inclined at about 15°, is done with looping on the front edge between the front diopter 15 and the edge 18 of the narrow edge 17 such that in this connecting zone, the rear face of the peduncles 5 and struts 13 is found in the extension of the concave neck mold 19.

The purpose of this arrangement is to produce a transition in softness from the embedding of the rear portion of the implant in the capsular bag by orienting the tension forces that are exerted on the bag in parallel to the rear face of the peduncles and struts, which has the effect of preventing the formation of folds on the edge 18 as is illustrated diagrammatically in FIG. 8c. This figure, in the right portion, shows a connecting section without a neck mold 19 and, in the left portion, it shows a similar section with the neck mold 19. It is seen that without a neck mold, under the effect of a gradient importing pressure forces of the window to the rear diopter of the optic (arrows 20a), the capsule has a significant deflection that produces a tension-force resultant (arrow 20b) that is oriented out of parallel with respect to the rear face of the peduncle 5. In the presence of the neck mold 19, the pressure forces of the window (arrows 20a′) gradually decrease from the edge 16 of the rear diopter to the edge 18, such that the capsule takes on the concavity of the neck mold 19 and such that the tension-force resultant (arrow 20b′) is oriented in parallel to the rear face of the peduncle 5.

In FIGS. 8a and 8b, the behavior of the lens according to the invention was shown diagrammatically under the effect of the compression of the support portion of its haptic portion during its installation in the capsular bar. The force that is symbolized by the arrow 21 produces a projection of the optical portion toward the rear on the order of 0.3 mm for a reduction of the overall diameter of the lens of 13 mm to 10.5 mm. The effect of this projection is to flatten the rear diopter against the wall of the capsular bag in which it is embedded thanks to the edge 16, thereby closely copying the shape of the neck mold 19. This produces an effective barrier against the progression of the epithelial cells in the direction of the wall of the capsular bag.

The curvature radius of the neck mold 19 is preferably on the order of 0.2 to 0.6 mm, and its maximum depth is 0.1 mm.

The embodiments that are described above relate to an intraocular lens with a capsular bag. The invention, however, covers all the types of intraocular lenses, whether they are implanted in or outside of the capsular bag.

1. Intraocular lens characterized by the fact that the support portion (4) of its haptic portion (3), elevation view, has the shape of a flat semi-circular closed loop with a constant thickness at rest, the proximal portion of said loop originating on the distal portion of a connecting peduncle (5) that is oriented essentially tangentially to the periphery of the optical portion (2) parallel to the vertical diameter (d1) of said optical portion, whereby the support portion (4) of said haptic portion (3) forms an angle of about 30° with the horizontal diameter (d2) of said optical portion (2).

2. Intraocular lens according to claim 1, wherein the support portion (4) of the haptic portion (3) has a general arc shape with an aperture angle of said arc of between 30° and 60°, and preferably 40°.

3. Intraocular lens according to claim 1, wherein the support portion (4) of the haptic portion (2) has a constant section over its entire length.

4. Intraocular lens according to claim 1, wherein the current section of thread or strip forming the loop of the support portion (4) is between 0.05 and 0.50 mm², preferably 0.20 mm².

5. Intraocular lens according to claim 1, wherein the width (11) and the thickness (12) of the thread or strip forming the loop of the support portion (4) of the haptic portion (3) are between 0.2 and 0.8 mm, preferably 0.4 mm.

6. Intraocular lens according to claim 5, wherein the thickness (12) of the thread or strip forming the loop of the support portion (4) of the haptic portion (3) is at least equal to or is greater than its width (11).

7. Intraocular lens according to claim 1, wherein the height (8) of the support portion (4) of its haptic portion (3) is between 0.6 and 3 mm, preferably 1.4 mm, and wherein its length (9) is at least equal to twice its width.

8. Intraocular lens according to claim 1, wherein the connecting portion (5) of the support portion (4) on the optical portion (2) extends, plan view, in a general direction that is virtually parallel to the vertical diameter (d1) of the optical portion (2) and wherein its outside edge (5′) is essentially tangential to the periphery of the optical portion (2).

9. Intraocular lens according to claim 8, wherein the connecting portion (5) is connected to the periphery of the optical portion (2) over a relatively small angular distance relative to the circumference of the lens, preferably on the order of 15° to 20°.

10. Intraocular lens according to claim 8, wherein the connecting portion (5) is connected to the periphery of the optical portion (2) by forming with the latter an angle of about 15° relative to the optical plane.

11. Intraocular lens according to claim 8, wherein it comprises a strut (13) that is placed under the support portion (4) of its haptic portion (3), near the proximal end of said support portion (4).

12. Intraocular lens according to claim 11, wherein the strut (13) is placed essentially parallel to the connecting portion (5).

13. Intraocular lens according to claim 8, wherein the diameter of the rear diopter (14) of its optical portion (2) is smaller than the diameter of the body (15) and wherein the peripheral edge (16) of the rear diopter (14) is connected to the narrow peripheral edge (17) of the optical portion (2) by a concave neck mold (19).

14. Intraocular lens according to claim 13, wherein the implantation of the connecting portion (5) and/or the strut (13) on the periphery of the optical portion (2) is carried out with looping on the edge of the front diopter (15) such that the rear face of said connecting portion (5) and/or strut (13) is found in the extension of the concave neck mold (19).

15. Intraocular lens according to claim 13, wherein the curvature radius of the neck mold 19 is on the order of 0.2 to 0.6 mm, and its maximum depth is 0.1 mm.

16. Intraocular lens according to claim 2, wherein the support portion (4) of the haptic portion (2) has a constant section over its entire length.

17. Intraocular lens according to claim 9, wherein the connecting portion (5) is connected to the periphery of the optical portion (2) by forming with the latter an angle of about 15° relative to the optical plane.

18. Intraocular lens according to claim 9, wherein the diameter of the rear diopter (14) of its optical portion (2) is smaller than the diameter of the body (15) and wherein the peripheral edge (16) of the rear diopter (14) is connected to the narrow peripheral edge (17) of the optical portion (2) by a concave neck mold (19).

19. Intraocular lens according to claim 10, wherein the diameter of the rear diopter (14) of its optical portion (2) is smaller than the diameter of the body (15) and wherein the peripheral edge (16) of the rear diopter (14) is connected to the narrow peripheral edge (17) of the optical portion (2) by a concave neck mold (19).

20. Intraocular lens according to claim 14, wherein the curvature radius of the neck mold 19 is on the order of 0.2 to 0.6 mm, and its maximum depth is 0.1 mm.
